(19) United States
(12) Patent Application Publication
(10) Pub. No.: US 2010/0196305 A1
(43) Pub. Date: Aug. 5, 2010

(54) SULFONE POLYMER COMPOSITIONS

(76) Inventors: Pradeep K. Dhal, Westford, MA (US); Stephen Randall Holmes-Farley, Arlington, VA (US); Chad C. Huval, Somerville, MA (US); Steven C. Polomoscanik, Bedford, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 12/449,974

(22) PCT Filed: Mar. 5, 2008
(86) PCT No.: PCT/US08/02900

§ 371 Date: Mar. 4, 2010

Related U.S. Application Data
(60) Provisional application No. 60/905,595, filed on Mar. 8, 2007.

(51) Int. Cl. A61K 31/795; A61P 3/00

(52) U.S. Cl. 424/78.08

(57) ABSTRACT

Sulfone-containing copolymers and copolymer networks and compositions including sulfone-containing copolymers and copolymer networks may be used to bind target ions, such as phosphorous-containing compounds in the gastrointestinal tract of animals. In some cases, sulfone-containing copolymers and copolymer networks may be derived from a multi-amine monomer and a multifunctional sulfonyl-containing monomer comprising two or more amine-reactive groups.

### FIELD OF THE INVENTION

This invention relates to polymers, copolymers, polymer networks and/or copolymer networks for binding target ions, and more specifically relates to pharmaceutically acceptable compositions, polymers, copolymers, polymer networks and/or copolymer networks for binding target ions.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### BRIEF SUMMARY OF THE INVENTION

In one aspect, the present invention relates to polymers, copolymers, polymer networks, copolymer networks and/or pharmaceutical compositions comprising the same. The polymers and copolymers can be crosslinked to form polymer networks and copolymer networks respectively. Compositions can comprise polymers or residues thereof, copolymers or residues thereof, polymer networks and/or copolymer networks. Several embodiments of the invention, including this aspect of the invention, are described in further detail as follows. Generally, each of these embodiments can be used in various and specific combinations, and with other aspects and embodiments unless otherwise stated herein.

In addition to the polymers, copolymers, polymer networks and copolymer networks of the present invention as described herein, other forms of the polymers, copolymers, polymer networks and copolymer networks are within the scope of the invention including pharmaceutically acceptable salts, solvates, hydrates, prodrugs, polymorphs, clathrates, and isotopic variants and mixtures thereof of polymers, copolymers, polymer networks and/or copolymer networks.

In addition, polymers, copolymers, polymer networks, and copolymer networks of the invention may have optical centers, chiral centers or double bonds and the polymers, copolymers, polymer networks and copolymer networks of the present invention include all of the isomeric forms of these polymers, copolymers, polymer networks and copolymer networks, including optically pure forms, racemates, diastereomers, enantiomers, tautomers and/or mixtures thereof.

The invention provides methods of treating an animal, including a human. The method generally involves administering an effective amount of a polymer, copolymer, polymer network and/or a copolymer network or a composition (e.g., a pharmaceutical composition) comprising the same as described herein.

In some embodiments, the invention is, consists essentially of, or comprises a copolymer or residue thereof and/or a copolymer network or a pharmaceutical composition comprising the same, where the copolymer is derived from two or more monomers or comprises a residue of two or more monomers where the monomers comprise a multi-amine monomer and a multifunctional sulfonyl-containing monomer comprising two or more amine-reactive groups. In some embodiments at least one of the amine-reactive groups comprises a vinyl group, such as for example, an α,β-unsaturated sulfonyl group.

In one embodiment, the invention is, consists essentially of, or comprises a copolymer or residue thereof and/or a copolymer network that is derived from at least one monomer represented by Formula I and at least one monomer represented by Formula II as follows:

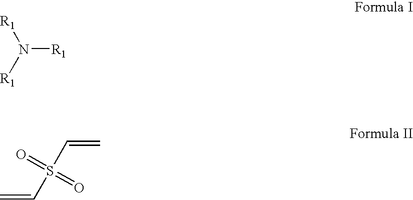

where R₁ independently represents a hydrogen radical, —R or —R—N(H)₂₋ₘ—R—N(H)₂₋ₙ—(R—NH₂)ₙ)ₘ or R₁ and another R₁ combine to form a heterocyclic ring, such as for example a heterocyclic ring comprising 1-4 heteroatoms, such as 1, 2, 3 or 4 heteroatoms, such as 1-4 nitrogen atoms, where the ring also includes 1-10 carbon atoms, such as 1, 2, 3, 4, 5, 6, 7, 8, or 9 carbon atoms; n and m independently represents an integer from 0 to 2, such as 0, 1 or 2, preferably either n or m is 1; R independently represents a branched or unbranched, substituted or unsubstituted alkyl radical, for example a C₁ to C₂₀ radical such as a C₁, C₂, C₃, C₄, C₅, or C₆ radical, with the proviso that at least one R₁ is not a hydrogen radical or —R.

Another aspect of the invention is a pharmaceutical composition comprising one or more polymers, copolymers, polymer networks and/or copolymer networks of the present invention and at least one pharmaceutically acceptable excipient. The polymers, copolymers, polymer networks and/or copolymer networks described herein have several therapeutic applications. For example, they are useful in removing compounds or ions such as anions, for example phosphorous-containing compounds or phosphorous containing ions such as organophosphates and/or phosphates, from the gastrointestinal tract, such as from the stomach, small intestine and/or large intestine. In some embodiments, the polymers, copolymers, polymer networks, and/or copolymer networks are used in the treatment of phosphate imbalance disorders and renal diseases.

In some embodiments, the invention comprises polymers and/or copolymers described herein are formed from one or more monomers using a one pot or single step synthesis and polymer networks, copolymer networks and/or pharmaceutical compositions formed therefrom.

In yet another aspect, the polymers, copolymers, polymer networks and/or copolymer networks described herein are useful for removing other solutes, such as chloride, bicarbonate, and/or oxalate containing compounds or ions. Polymers, copolymers, polymer networks and/or copolymer networks removing oxalate compounds or ions find use in the treatment of oxalate imbalance disorders. Polymers, copolymers, polymer networks and/or copolymer networks removing chloride compounds or ions find use in treating acidosis, for example. In some embodiments, the polymers, copolymers, polymer networks and/or copolymer networks are useful for removing bile acids and related compounds.

The invention further provides compositions containing any of the polymers, copolymers, polymer networks and/or copolymer networks described herein where the polymers, copolymers, polymer networks and/or copolymer networks are in the form of particles and where the particles are encased in one or more shells.

In another aspect, the invention provides pharmaceutical compositions. In one embodiment, the pharmaceutical composition contains one or more polymers, copolymers, polymer networks and/or copolymer networks of the invention and a pharmaceutically acceptable excipient. In some embodiments, the composition is a liquid formulation in which the polymer, copolymer, polymer network and/or copolymer network is dispersed in a liquid vehicle, such as water, and suitable excipients. In some embodiments, the invention provides a pharmaceutical composition comprising a polymer, copolymer, polymer network and/or copolymer network for binding a target compound or ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the polymer, copolymer, polymer network and/or copolymer network is an organophosphate and/or phosphate. In some embodiments the polymer, copolymer, polymer network and/or copolymer network is more than about 50% of the weight of the tablet. In some embodiments, the tablet is of cylindrical shape with a diameter of from about 12 mm to about 28 mm and a height of from about 1 mm to about 8 mm and the amine polymer comprises more than 0.6 to about 2.0 gm of the total weight of the tablet.

In some of the compositions of the invention, the excipients are chosen from the group consisting of sweetening agents, binders, lubricants, and disintegrants. Optionally, the polymer, copolymer, polymer network and/or copolymer network is present as particles of less than about 80 μm mean diameter. In some of these embodiments, the sweetening agent is selected from the group consisting of sucrose, mannitol, xylitol, maltodextrin, fructose, and sorbitol, and combinations thereof.

In some embodiments, the invention provides copolymers, copolymer networks, or compositions that comprise a copolymer or residue thereof, where the copolymer is derived from two or more comonomers comprising at least one multi-amine or residue thereof and at least one vinyl sulfonyl-containing monomer or residue thereof.

In some embodiments, polymers and/or copolymers of the invention may comprise hyperbranched polymers. In some embodiments, polymers and/or copolymers of the invention include polymers and/or copolymers where from 10-95% of the amine groups in the polymer and/or copolymer comprise secondary amine groups. In other embodiments, polymers and/or copolymers of the invention may have a degree of branching of from 0.10 to 0.95. In other embodiments, polymers and/or copolymers of the invention have a polydispersity of greater than 1.2. In some embodiments, polymers and/or copolymers of the invention may be branched and may be characterized by a plot of log (Mᵥ) versus log (η) that has no maximum, where Mᵥ represents the viscosity averaged molecular weight of the polymer and η represents the intrinsic viscosity of the polymer. In other embodiments, polymers and/or copolymers of the invention include polymers and or copolymers where greater than 10% and less than 90% of the non-terminal, non-amido amine groups in the polymer or copolymer are tertiary amines. In some embodiments, the invention comprises polymer networks or copolymer networks formed from polymers or copolymers having any one or more of these properties, methods of treatment, for example treatment of hyperphosphatemia comprising administering an effective amount of one or more polymers, copolymers, polymer networks, copolymer networks or compositions (e.g., pharmaceutical compositions) comprising the same to an animal in need thereof, where the polymers or copolymers have any one or more of these properties.

In still other embodiments, a polymer network and/or copolymer network may include two or more polymers or copolymers, where at least one of the polymers or copolymers is a derived from monomers according to Formulas I and II, that may be linked or crosslinked to form a polymer network or copolymer network. For example, in some embodiments a polymer network or copolymer network may comprise a residue of two or more polymers or copolymers according to the invention and a residue of one or more crosslinking agents.

### DETAILED DESCRIPTION OF THE INVENTION

In one aspect, the invention is, consists essentially of, or comprises a hyperbranched polymer or residue thereof, a hyperbranched copolymer or residue thereof, a hyperbranched polymer network and/or a hyperbranched copolymer network or a pharmaceutical composition comprising the same.

In another aspect, the present invention provides copolymers, copolymer networks that comprise said copolymers or residues thereof, compositions (e.g., pharmaceutical compositions) that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymer is derived from, or comprises a residue of, a multi-amine monomer and a multifunctional sulfonyl-containing monomer comprising two or more amine-reactive groups where the amine-reactive groups may be, for example, vinyl groups, acid groups, ester groups and/or combinations thereof. The amine-reactive groups may react with the multi-amine via any suitable reaction, for example via a condensation or polycondensation reaction or via an alkylation reaction. In some embodiments, the reaction may include a combination of different reactions, such as a combination of alkylation and condensation reactions. In some embodiments the reaction or reactions may be controlled by any suitable means including, for example, choice of solvent, temperature, concentration of reactants, protection using protecting groups, pH and/or any other suitable methods.

In some embodiments, the multifunctional sulfonyl-containing monomer is a vinyl sulfonyl-containing monomer and may be selected from the group consisting of:

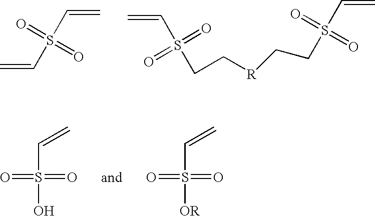

wherein R independently represents a branched or unbranched, substituted or unsubstituted alkyl or aryl radical.

In one aspect, the present invention provides copolymers, copolymer networks that comprise said copolymers or residues thereof, compositions (e.g., pharmaceutical compositions) that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymers are derived from comonomers represented by the following Formulas I and II:

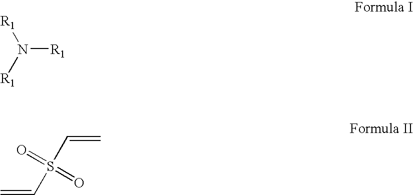

where R₁ independently represents a hydrogen radical, —R or

—R—N(H)₂₋ₘ—(R—N(H)₂₋ₙ—(R—NH₂)ₙ)ₘ or R₁ and another R₁ combine to form a heterocyclic ring, such as for example a heterocyclic ring comprising 1-4 heteroatoms, such as 1, 2, 3 or 4 heteroatoms, such as 1-4 nitrogen atoms, where the ring also includes 1-10 carbon atoms, such as 1, 2, 3, 4, 5, 6, 7, 8, or 9 carbon atoms; n and m independently represents an integer from 0 to 2, such as 0, 1 or 2, preferably either n or m is 1; R independently represents a branched or unbranched, substituted or unsubstituted alkyl radical, for example a C₁ to C₂₀ radical such as a C₁, C₂, C₃, C₄, C₅, or C₆ radical, with the proviso that at least one R₁ is not a hydrogen radical or —R.

In one aspect, the present invention provides copolymers, copolymer networks that comprise said copolymers or residues thereof, compositions (e.g., pharmaceutical compositions) that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymers comprise a residue of one or more multi-amine compounds and a residue of one or more vinyl sulfonyl-containing compounds. In some embodiments, the multi-amine monomer comprises at least one secondary amine.

In some embodiments, polymers and/or copolymers of the invention include polymers and or copolymers where from 10-95%, for example 10-75%, 25%-75%, 30%-60%, such as 20%, 25%, 30%, 35%, 40%, 45%, 50%, or 55% of the amine groups in the polymer or copolymer comprise secondary amine groups. In other embodiments, polymers and/or copolymers of the invention include polymers and or copolymers where greater than 10% and less than 90%, for example, from 15%-85%, 20%-80%, 30%-70%, such as 35%, 40%, 45%, 50%, 55%, 60% or 65% of the non-terminal, non-amido amine groups in the polymer or copolymer are tertiary amines. In other embodiments, polymers and/or copolymers of the invention may have a degree of branching of from 0.10 to 0.95, such as from 0.25-0.75, 0.30-0.60, or such as a degree of branching of 0.2, 0.25, 0.3, 0.35, 0.4, 0.45, 0.5, 0.55 which, in some embodiments may be calculated according to the following formula:

$$Degree of Branching = \frac{{N}_{p} + {N}_{t}}{{N}_{p} + {N}_{t} + {N}_{s}}$$

where

Nₚ=the number of primary amine units in the polymer (e.g., —NH₂ units);

Nₜ=the number of tertiary amine units in the polymer (e.g.,

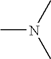

units; and

Nₛ=the number of secondary amine units in the polymer

(e.g.,

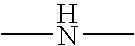

and

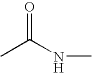

units).

In other embodiments, polymers and/or copolymers of the invention have a polydispersity of greater than 1.2, for example greater than 1.3, 1.4, 1.5, 1.75, 2.0, 2.5 or even greater than 3.0, such as from 1.2-6, such as 1.5-5 or 2-4. In some embodiments, polymers and/or copolymers of the invention may be branched and may be characterized by a plot of log (Mᵥ) versus log (η) that has no maximum, where Mᵥ represents the viscosity averaged molecular weight of the polymer or copolymer and η represents the intrinsic viscosity of the polymer or copolymer. For example, in some embodiments, polymers and copolymers of the invention where the following equation is true: d(log(η))/d(log(Mᵥ))≠0.

In some embodiments, polymers and/or copolymers of the invention may have random, variable length branching. For example, polymers or copolymers of the invention may exhibit branching that does not conform to a regular or easily predictable or quantifiable pattern of occurrence or length and instead results from essentially random molecular interactions that may be driven by a wide variety of different variables such as, for example, monomer concentration, reactivity, pH, solvent, temperature, charge-charge interactions, catalysis, order of addition, and any other reaction parameters.

As used herein, unless otherwise stated, the term “derived from” is understood to mean: produced or obtained from another substance by chemical reaction, especially directly derived from the reactants, for example a polymer or copolymer may be derived from the reaction of a multi-amine compound and one or more vinyl sulfonyl-containing compounds. Additionally, a polymer or copolymer that is reacted with a linking agent, such as a crosslinking agent results in an polymer network or a copolymer network that is derived from the polymer or copolymer and the linking agent.

In one aspect, the present invention provides copolymers, copolymer networks that comprise said copolymers or residues thereof, compositions (e.g., pharmaceutical compositions) that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymers are derived from comonomers represented by the following Formulas I and II:

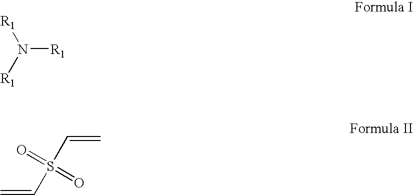

where R₁ independently represents a hydrogen radical, —R or —R—N(H)₂₋ₘ—(R—N(H)₂₋ₙ—(R—NH₂)ₙ)ₘ or R₁ and another R₁ combine to form a heterocyclic ring, such as for example a heterocyclic ring comprising 1-4 heteroatoms, such as 1, 2, 3 or 4 heteroatoms, such as 1-4 nitrogen atoms, where the ring also includes 1-10 carbon atoms, such as 1, 2, 3, 4, 5, 6, 7, 8, or 9 carbon atoms; n and m independently represents an integer from 0 to 2, such as 0, 1 or 2, preferably either n or m is 1; R independently represents a branched or unbranched, substituted or unsubstituted alkyl radical, for example a C₁ to C₂₀ radical such as a C₁, C₂, C₃, C₄, C₅, or C₆ radical, with the proviso that at least one R₁ is not a hydrogen radical or —R where the copolymer is hyperbranched.

In one aspect, the present invention provides copolymers, copolymer networks that comprise said copolymers or residues thereof, compositions (e.g., pharmaceutical compositions) that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymers are derived from comonomers represented by the following Formulas I and II:

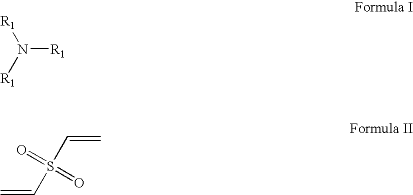

where R₁ independently represents a hydrogen radical, —R or —R—N(H)₂₋ₘ—(R—N(H)₂₋ₙ—(R—NH₂)ₙ)ₘ or R₁ and another R₁ combine to form a heterocyclic ring, such as for example a heterocyclic ring comprising 1-4 heteroatoms, such as 1, 2, 3 or 4 heteroatoms, such as 1-4 nitrogen atoms, where the ring also includes 1-10 carbon atoms, such as 1, 2, 3, 4, 5, 6, 7, 8, or 9 carbon atoms; n and m independently represents an integer from 0 to 2, such as 0, 1 or 2, preferably either n or m is 1; R independently represents a branched or unbranched, substituted or unsubstituted alkyl radical, for example a C₁ to C₂₀ radical such as a C₁, C₂, C₃, C₄, C₅, or C₆ radical, with the proviso that at least one R₁ is not a hydrogen radical or —R, where the copolymer has one or more of the following characteristics:
a degree of branching of from 0.10 to 0.95;
from 10-95% of the nitrogen atoms in the copolymer are the nitrogen in a secondary amine moiety;
a polydispersity greater than about 1.2;
random, variable length branching;
greater than 10% and less than 90% of non-terminal, non-sulfonamido amine groups in said copolymer comprise tertiary amines;
when branched, an intrinsic viscosity that has no maximum (versus viscosity averaged molecular weight).

In some embodiments, a polymer network or copolymer network comprises a residue of a polymer or copolymer as described herein and a residue of one or more crosslinking agents. In some embodiments, the crosslinking agent comprises an epihalohydrin such as, for example, epichlorohydrin.

In some embodiments, the present invention provides copolymers, copolymer networks that comprise said copolymers or residues thereof, compositions that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymer is derived from a multi-amine monomer and a vinyl sulfonyl-containing monomer and where the copolymer has one or more of the following characteristics:
a degree of branching of from 0.10 to 0.95;
from 10-95% of the nitrogen atoms in the copolymer are the nitrogen in a secondary amine moiety;
a polydispersity greater than about 1.2;
random, variable length branching;
greater than 10% and less than 90% of non-terminal, non-sulfonamido amine groups in said copolymer comprise tertiary amines;
when branched, an intrinsic viscosity that has no maximum (versus viscosity averaged molecular weight).

In some embodiments, the present invention provides copolymers, copolymer networks that comprise said copolymers or residues thereof, compositions that comprise copolymers and/or copolymer networks, and methods for removing a compound or ion, such as a phosphorous-containing compound or a phosphorous-containing ion (e.g. phosphate) from the gastrointestinal tract of an animal by administering an effective amount of a copolymer or copolymer network, where the copolymer comprises a residue of one or more multi-amine compounds and a residue of one or more vinyl sulfonyl-containing compounds, where the copolymer has one or more of the following characteristics:
a degree of branching of from 0.10 to 0.95;
from 10-95% of the nitrogen atoms in the copolymer are the nitrogen in a secondary amine moiety;
a polydispersity greater than about 1.2;
random, variable length branching;
greater than 10% and less than 90% of non-terminal, non-sulfonamido amine groups in said copolymer comprise tertiary amines;
when branched, an intrinsic viscosity that has no maximum (versus viscosity averaged molecular weight).

In some embodiments, the multi-amine monomer described herein comprises at least one secondary amine. Examples of such multi-amine monomers include:

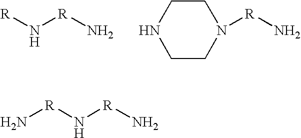

and combinations thereof, where R independently represents a branched or unbranched, substituted or unsubstituted alkyl radical, for example a C₁ to C₂₀ radical such as a C₁, C₂, C₃, C₄, C₅, or C₆ radical.

In some embodiments, one or more amine groups of the multi-amine monomer or a compound according to Formula I described herein may be protected with a protecting group, such as, for example, protected with a tert-butyloxycarbonyl group.

In some embodiments, polymers and copolymers of the invention may comprise more than one multi-amine or residue thereof. In some embodiments, polymers and copolymers of the invention may be reacted post polymerization with a further multi-amine, for example by reacting any remaining amine-reactive groups in the polymer or copolymer with a multi-amine.

In some embodiments the multi-amine may be selected from the group consisting of:

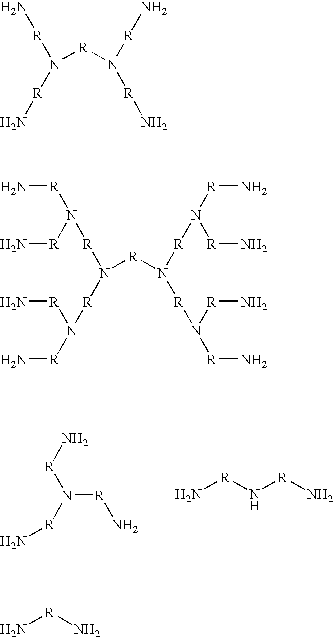

and combinations thereof, wherein R independently represents a branched or unbranched, substituted or unsubstituted alkyl radical such as, for example, compounds such as:

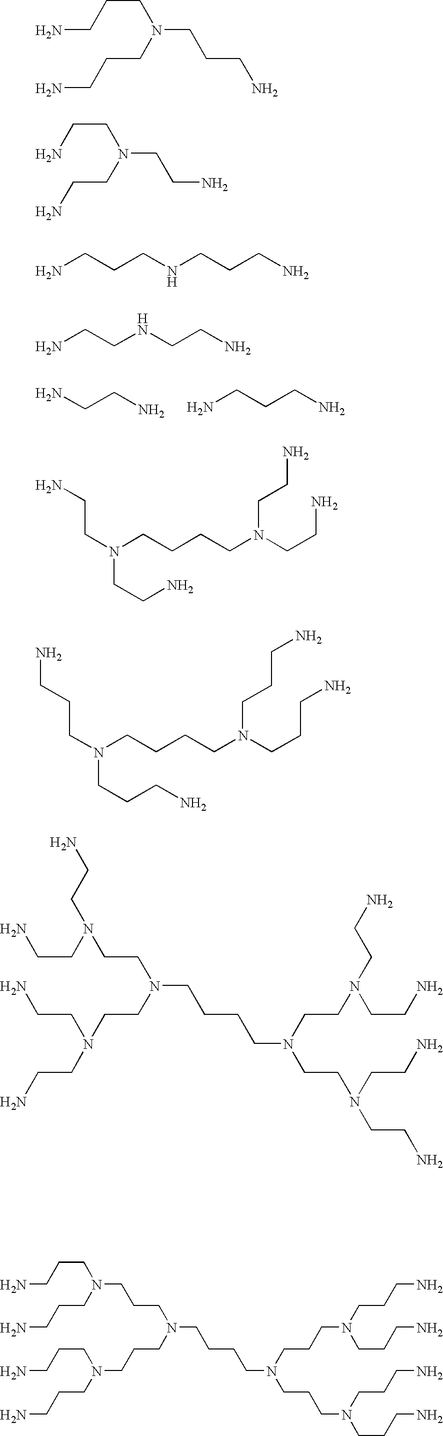

and combinations thereof.

In some embodiments, the multi-amine according to the invention may comprise from 2 to 20 amine groups and may comprise at least one, such as 2, 3, 4, 5, 6, 7, 8, 9, 10, 11, 12, 13, 14, 15, 16, 17, 18, 19, or 20 secondary amine groups. In some embodiments, the multi-amine according to the invention comprises a compound according to Formula I. In some embodiments the multi-amine may have from 2-20, such as 3, 4, 5, 6, 7, 8, 9, 10, 11, 12, 13, 14, 15, 16, 17, 18 or 19 terminal amine groups.

In some embodiments, a polymer network or copolymer network comprises a residue of a polymer or copolymer as described herein and a residue of one or more crosslinking agents. In some embodiments, the crosslinking agent comprises epichlorohydrin.

In some embodiments, a method of making copolymers of the invention can include any suitable method such as addition of a multi-amine to a compound comprising two or more amine-reactive groups, such as a vinyl sulfonyl-containing monomer, in a reactor and heating the mixture. In some embodiments, the vinyl sulfonyl-containing monomer may be divinyl sulfone. In some embodiments the mixture may be heated to greater than 25° C., for example 30° C., 35° C., 37° C., 40° C., 45° C., 50° C. or higher. In some embodiments, the mixture may be heated from 1 hour to several days, such as 1-7 days, such as from 2-6 days or 24, 48, 72 or 96 hours. The resulting copolymer may be purified using any suitable method, such as precipitation and washing, or dialyzation. The copolymer may then be dried under vacuum or lyophilized to yield the desired copolymer.

The copolymer prepared above then be subsequently crosslinked using any suitable method. For example, the copolymer may be mixed with a crosslinking agent, such as for example epichlorohydrin, in a suitable solvent, such as, for example, water and stirred. In some embodiments, the crosslinking agent may be added in one or more aliquots such as 1-10 aliquots, such as 2-8 or 3-5 aliquots. In some embodiments, the solution may be stirred and heated for 1 hour to 5 days, such as 1, 2, 3, 4 or 5 days. A gel may form and may be cured, broken, resuspended and washed one or more times and then dried, such as in a forced air oven or via lyophilization. In some embodiments, washing may include adjustment of the pH of the material.

In some embodiments, the invention is a method for reducing blood phosphate levels by 5-100% in a patient in need thereof, the method comprising administering a therapeutically effective amount of one or more polymers, copolymers, polymer networks and/or copolymer networks of the invention or a composition comprising one or more one or more polymers, copolymers, polymer networks and/or copolymer networks of the invention to the patient. In some embodiments, the invention is a method for reducing urinary phosphorous by 5-100% in a patient in need thereof, the method comprising administering a therapeutically effective amount of one or more polymers, copolymers, polymer networks and/or copolymer networks of the invention or a composition comprising one or more one or more polymers, copolymers, polymer networks and/or copolymer networks of the invention to the patient.

In some embodiments, the invention is a method of treating a phosphate imbalance disorder such as hyperphosphatemia comprising administering a therapeutically effective amount of one or more polymers, copolymers, polymer networks and/or copolymer networks of the invention or a composition comprising one or more one or more polymers, copolymers, polymer networks and/or copolymer networks of the invention to a patient in need thereof.

In some embodiments, the composition is a mixture of more than one polymer, copolymer, polymer network and/or copolymer network of the invention, for example 2-20 such as 2, 3, 4, 5, 6, 7, 8, 9 or 10 polymers, copolymers, polymer networks and/or copolymer networks of the invention.

In some embodiments, the invention comprises a polymer, copolymer, polymer network and/or copolymer network of the invention derived from a multi-amine compound that is a mixture of multi-amine compounds, a pharmaceutical composition comprising such a polymer, copolymer, polymer network and/or copolymer network, or a method of using the same in a therapeutically effective amount to remove a compound or ion, such as a phosphorous-containing compound or a phosphorous-containing ion (e.g. phosphate), from the gastrointestinal tract of an animal.

Other embodiments of the invention include pendant polymers formed with polymers, copolymers polymer networks and/or copolymer networks as pendant groups on a polymer or polymerized backbone of a polymer. Such pendant polymers may be formed by adding one or more polymerizable groups to one or more amine groups on a polymer, copolymer, polymer network and/or copolymer network to form a pendant monomer and then subsequently polymerizing the polymerizable group to form a pendant polymer comprising a polymer, copolymer, polymer network and/or copolymer network. A schematic example of such an addition follows [it should be noted in the following that a polymer, copolymer, polymer network and/or copolymer network designated as “AC” is intended to represent a polymer, copolymer, polymer network and/or copolymer network or residue thereof, of the invention, with one of its amine groups depicted for purposes of illustrating how a polymerizable group may be added to the polymer, copolymer, polymer network and/or copolymer network]:

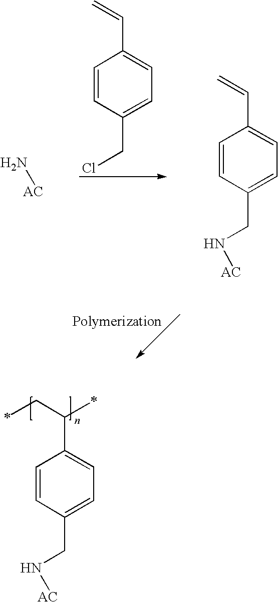

Non-limiting examples of other polymerizable groups that may be used with polymers, copolymers, polymer networks and/or copolymer network according to embodiments of the invention include:

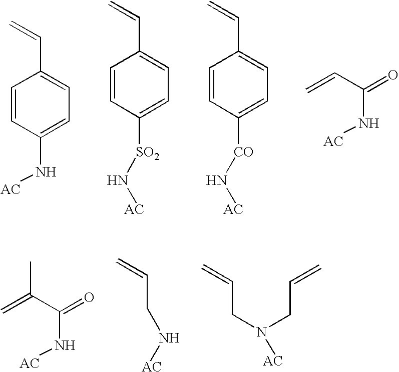

One or more polymerizable groups may be added to each AC and thus it is possible to have mixtures of pendant monomers having various pendant ACs having differing numbers of polymerizable groups. In addition, the pendant polymers made in this fashion may be modified, crosslinked, formed into a network or substituted post polymerization. Such modification may be performed for any number of reasons, including to improve efficacy, tolerability or reduce side effects.

Pendant monomers may also be formed by addition of ACs to amine-reactive polymers by reacting one or more amine groups of the ACs with one or amine-reactive groups on the amine-reactive polymers. Examples of some amine reactive polymers include:

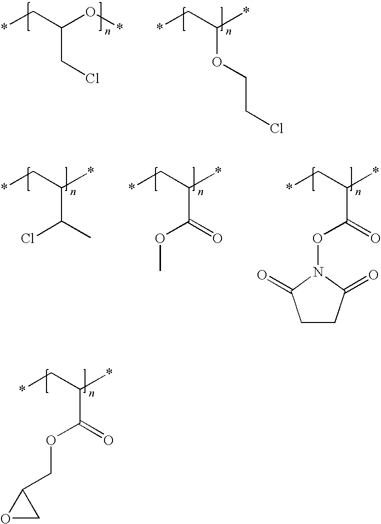

The ACs or pendant monomers may also serve as multifunctional monomers to form polymers. For example, when the ACs or the polymers formed from the pendant monomers are crosslinked, the crosslinking reaction may be carried out either in solution of bulk (i.e. using the neat amine and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), dimethylformamide, dimethylsulfoxide, acetone, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, emulsion, precipitation techniques, polymerization in aerosol or using bulk polymerization/crosslinking methods and size reduction processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amine and crosslinking agents in a droplet separate phase.

Polymers and copolymers, pendant monomers and pendant polymers of the invention may be copolymerized with one or more other monomers or oligomers or other polymerizable groups, may be crosslinked, may have crosslinking or other linking agents or monomers within the polymer backbone or as pendant groups or may be formed or polymerized to form a polymer network or mixed or copolymer network comprising: polymer or copolymers or residues thereof, pendant monomers or residues thereof, crosslinking agent or residues thereof, or other linking agents or residues thereof. The network may include multiple connections between the same or different molecules that may be direct or may include one or more linking groups such as crosslinking agents or other linking agents such as monomers or oligomers or residues thereof.

Non-limiting examples of comonomers which may be used alone or in combination include: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyle, methallyl monomers and combinations thereof. Functionalized versions of these monomers may also be used. Additional specific monomers or comonomers that may be used in this invention include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N—N-butylmethacrylamide, N-methylolmethacrylamide, N-ethylolmethacrylamide, N-tert-butylacrylamide, N—N-butylacrylamide, N-methylolacrylamide, N-ethylolacrylamide, 4-acryloylmorpholine, vinyl benzoic acid (all isomers), diethylaminostyrene (all isomers), α-methylvinyl benzoic acid (all isomers), diethylamino α-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylformamide, N-vinyl acetamide, allylamine, methallylamine, allylalcohol, methyl-vinylether, ethylvinylether, butylvinyltether, butadiene, isoprene, chloroprene, ethylene, vinyl acetate and combinations thereof.

In some embodiments, polymers and copolymers of the invention are crosslinked using crosslinking agents, and may not dissolve in solvents, and, at most, swell in solvents. The swelling ratio may be measured according to the procedure in the Test Methods section below and is typically in the range of about 1 to about 20; for example 2 to 10, 2.5 to 8, 3 to 6 such as less than 5, less than 6, or less than 7. In some embodiments, the polymers and copolymers may include crosslinking or other linking agents that may result in polymer or copolymer networks that do not form gels in solvents and may be soluble or partially soluble in some solvents.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of a polymer or copolymer described herein.

Examples of crosslinking agents that are suitable for synthesis of the polymers or copolymers of the present invention include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4h-chromene-2-carboxylate, bis[4-(2,3-epoxy-propylthio)phenyl]-sulfide, 1,3-bis(3-glycidoxypropyl)tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7.3.3.15,11]heptasiloxane, 4,4′-methylenebis(N,N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate. When the crosslinking agent is an alkylhalide compound, a base can be used to scavenge the acid formed during the reaction. Inorganic or organic bases are suitable. NaOH is preferred. The base to crosslinking agent ratio is preferably between about 0.5 to about 2.

In some embodiments, the crosslinking agents may be introduced into the polymerization reaction in an amount of from 0.5 to 25 wt. % based on the total weight of the amine polymer or polymer, such as from about 2 to about 15 wt. %, from about 2 to about 12 wt. %, from about 3 to about 10 wt. %, or from about 3 to about 6 wt. %, such as 2, 3, 4, 5, 6 wt %. The amount of crosslinking agent necessary may depend on the extent of branching within the polymer or copolymer.

In some embodiments the weight averaged molecular weight of the polymers and copolymers, may be typically at least about 1000. For example, the molecular weight may be from about 1000 to about 1,000,000, such as about 2000 to about 750,000, about 3000 to about 500,000, about 5000 to about 250,000, about 10000 to about 100,000, such as from 15,000-80,000, 20,000-75,000, 25,000 to 60,000, 30,000 to 50,000, or 40,000-45,000.

The polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydrochloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy) hexane.

In some embodiments, any of the nitrogen atoms within the polymers, copolymers, polymer networks and/or copolymer networks according to embodiments of the invention may optionally be quaternized to yield the corresponding positively charged tertiary nitrogen group, such as for example, an ammonium or substituted ammonium group. Any one or more of the nitrogen atoms in the polymers, copolymers, polymer networks and/or copolymer networks may be quaternized and such quaternization, when present, is not limited to or required to include terminal amine nitrogen atoms. In some embodiments, this quaternization may result in additional network formation and may be the result of addition of crosslinking, linking or amine reactive groups to the nitrogen. The ammonium groups may be associated with a pharmaceutically acceptable counterion.

In some embodiments, polymers, copolymers, polymer networks and/or copolymer networks or residues thereof of the invention may be partially or fully quaternized, including protonated, with a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred ions include chlorides and carbonates.

In some embodiments, polymers, copolymers, polymer networks and/or copolymer networks or residues thereof of the invention may be protonated such that the fraction of protonated nitrogen atoms is from 1 to 25%, preferably 3 to 25%, more preferably 5 to 15%.

In one embodiment, a pharmaceutically acceptable polymer, copolymer, polymer network or copolymer network or residues thereof is a polymer, copolymer, polymer network and/or copolymer network or residues thereof in protonated form and comprises a carbonate anion. In one embodiment the pharmaceutically acceptable polymer, copolymer, polymer network and/or copolymer network is in protonated form and comprises a mixture of carbonate and bicarbonate anions.

In some embodiments, polymers, copolymers, polymer networks and/or copolymer networks of the invention are characterized by their ability to bind compounds or ions. Preferably the polymers, copolymers, polymer networks and/or copolymer networks of the invention bind anions, more preferably they bind organophosphates, phosphate and/or oxalate, and most preferably they bind organophosphates or phosphate. For illustration, anion-binding polymers, copolymers, polymer networks and/or copolymer networks and especially organophosphate or phosphate-binding polymers, copolymers, polymer networks and/or copolymer networks will be described; however, it is understood that this description applies equally, with appropriate modifications that will be apparent to those of skill in the art, to other ions, compounds and solutes. Polymers, copolymers, polymer networks and/or copolymer networks may bind an ion, e.g., an anion when they associate with the ion, generally though not necessarily in a noncovalent manner, with sufficient association strength that at least a portion of the ion remains bound under the in vitro or in vivo conditions in which the polymer is used for sufficient time to effect a removal of the ion from solution or from the body. A target ion may be an ion to which the polymers, copolymers, polymer networks and/or copolymer networks binds, and usually refers to the ion whose binding to the polymers, copolymers, polymer networks and/or copolymer networks is thought to produce the therapeutic effect of the polymer, copolymer, polymer network and/or copolymer network and may be an anion or a cation. A polymer, copolymer, polymer network and/or copolymer network of the invention may have more than one target ion.

For example, some of the polymers, copolymers, polymer networks and/or copolymer networks described herein exhibit organophosphate or phosphate binding properties. Phosphate binding capacity is a measure of the amount of phosphate ion a phosphate binder can bind in a given solution. For example, binding capacities of phosphate binders can be measured in vitro, e.g., in water or in saline solution, or in vivo, e.g., from phosphate urinary excretion, or ex vivo, for example using aspirate liquids, e.g., chyme obtained from lab animals, patients or volunteers. Measurements can be made in a solution containing only phosphate ion, or at least no other competing solutes that compete with phosphate ions for binding to the polymers, copolymers, polymer networks and/or copolymer networks. In these cases, a non interfering buffer may be used. Alternatively, measurements can be made in the presence of other competing solutes, e.g., other ions or metabolites, that compete with phosphate ions (the target solute) for binding to the polymers, copolymers, polymer networks and/or copolymer networks.

Ion binding capacity for a polymer, copolymer, polymer network and/or copolymer network may be measured as indicated in the Test Methods. Some embodiments have a phosphate binding capacity which can be greater than about 0.2, 0.5, 1.0, 1.5, 2.0, 2.5, 3.0, 3.5, 4.0, 5.0, 6.0, 8.0, 10.0, 12, 14, 16, 18 or greater than about 20 mmol/g. In some embodiments, the in vitro phosphate binding capacity of polymers, copolymers, polymer networks and/or copolymer networks or residues thereof of the invention for a target ion is greater than about 0.5 mmol/g, preferably greater than about 2.5 mmol/g, even more preferably greater than about 3 mmol/g, even more preferably greater than about 4 mmol/g, and yet even more preferably greater than about 6 mmol/g. In some embodiments, the phosphate binding capacity can range from about 0.2 mmol/g to about 20 mmol/g, such as about 0.5 mmol/g to about 10 mmol/g, preferably from about 2.5 mmol/g to about 8 mmol/g, and even more preferably from about 3 mmol/g to about 6 mmol/g. Phosphate binding may be measured according to the techniques described in the Test Methods section below.

In some embodiments, polymers, copolymers, polymer networks and/or copolymer networks and compositions of the invention may reduce urinary phosphorous of a patient in need thereof by 5-100%, such as 10-75%, 25-65%, or 45-60%. Some embodiments may reduce urinary phosphorous by greater than 10%, greater than 20%, greater than 30%, greater than 40%, greater than 45%, greater than 50% or greater than 60%. Reduction of urinary phosphorous may be measured according to the methods detailed in the Test Methods section below.

In some embodiments, polymers, copolymers, polymer networks and/or copolymer networks and compositions of the invention may reduce blood phosphate of a patient in need thereof by 5-100%, such as 10-75%, 25-65%, or 45-60%. Some embodiments may reduce blood phosphate levels by greater than 10%, greater than 20%, greater than 30%, greater than 40%, greater than 45%, greater than 50% or greater than 60%.

When crosslinked, some embodiments of the polymers or copolymers, e.g. polymer networks or copolymer networks, of the invention form a gel in a solvent, such as in a simulated gastrointestinal medium or a physiologically acceptable medium.

One aspect of the invention is core-shell compositions comprising a polymeric core and shell. In some embodiments, the polymeric core comprises the polymers, copolymers, polymer networks and/or copolymer networks described herein. The shell material can be chemically anchored to the core material or physically coated. In the former case, the shell can be grown on the core component through chemical means, for example by: chemical grafting of shell polymer to the core using living polymerization from active sites anchored onto the core polymer; interfacial reaction, i.e., a chemical reaction located at the core particle surface, such as interfacial polycondensation; and using block copolymers as suspending agents during the core particle synthesis.

In some embodiments, the interfacial reaction and use of block polymers are the techniques used when chemical methods are used. In the interfacial reaction pathway, typically, the periphery of the core particle is chemically modified by reacting small molecules or macromolecules on the core interface. For example, an amine containing ion-binding core particle is reacted with a polymer containing amine reactive groups such as epoxy, isocyanate, activated esters, halide groups to form a crosslinked shell around the core.

In another embodiment, the shell is first prepared using interfacial polycondensation or solvent coacervation to produce capsules. The interior of the capsule is then filled up with core-forming precursors to build the core within the shell capsule.

In some embodiments, using the block copolymer approach, an amphiphilic block copolymer can be used as a suspending agent to form the core particle in an inverse or direct suspension particle forming process. When an inverse water-in-oil suspension process is used, then the block copolymer comprises a first block soluble in the continuous oil phase and another hydrophilic block contains functional groups that can react with the core polymer. When added to the aqueous phase, along with core-forming precursor, and the oil phase, the block copolymer locates to the water-in-oil interface and acts as a suspending agent. The hydrophilic block reacts with the core material, or co-reacts with the core-forming precursors. After the particles are isolated from the oil phase, the block copolymers form a thin shell covalently attached to the core surface. The chemical nature and length of the blocks can be varied to vary the permeation characteristics of the shell towards solutes of interest.

When the shell material is physically adsorbed on the core material, well known techniques of microencapsulation such as solvent coacervation, fluidized bed spray coater, or multiemulsion processes can be used. One method of microencapsulation is the fluidized bed spray coater in the Wurster configuration. In yet another embodiment, the shell material is only acting temporarily by delaying the swelling of the core particle while in the mouth and esophagus, and optionally disintegrates in the stomach or duodenum. The shell is then selected in order to hinder the transport of water into the core particle, by creating a layer of high hydrophobicity and very low liquid water permeability.

In one embodiment the shell material carries negative charges while being in the milieu of use. Not being limited to one mechanism of action, it is thought that negatively charged shell material coated on anion-binding beads enhance the binding of small inorganic ions with a low charge density (such as phosphate) over competing ions with greater valency or size. Competing anions such as citrate, bile acids and fatty acids among others, may thus have a lesser relative affinity to the anion binding core possibly as a result of their limited permeability across the shell.

In some embodiments, shell materials are polymers carrying negative charges in the pH range typically found in the intestine. Examples include, but are not limited to, polymers that have pendant acid groups such as carboxylic, sulfonic, hydrosulfonic, sulfamic, phosphoric, hydrophosphoric, phosphonic, hydrophosphonic, phosphoramidic, phenolic, boronic and a combination thereof. The polymer can be protonated or unprotonated; in the latter case the acidic anion can be neutralized with pharmaceutically acceptable cations such as Na, K, Li, Ca, Mg, and NH₄.

In another embodiment the polyanion can be administered as a precursor that ultimately activates as a polyanion: for instance certain labile ester or anhydride forms of either polysulfonic or polycarboxylic acids are prone to hydrolysis in the acidic environment of the stomach and can convert to the active anions.

The shell polymers can be either linear, branched, hyperbranched, segmented (i.e. backbone polymer arranged in sequence of contiguous blocks of which at least one contains pendant acidic groups), comb-shaped, star-shaped or crosslinked in a network, fully and semi-interpenetrated network (IPN). The shell polymers are either random or block in composition and either covalently or physically attached to the core material. Examples of such shell polymers include, but are not limited to acrylic acid homopolymers or copolymers, methacrylic acid homopolymers or copolymers, and copolymers of methacrylate and methacrylic acid. Examples of such polymers are copolymers of methylmethacrylate and methacrylic acid and copolymers of ethylacrylate and methacrylic acid, sold under the tradename Eudragit (Rohm GmbH & Co. KG): examples of which include Eudragit L100-55 and Eudragit L100 (a methylmethacrylate-methacrylic acid (1:1) copolymer, Degussa/Rohm), Eudragit L30-D55, Eudragit S100-55 and Eudragit FS 30D, Eudragit S100 (a methylmethacrylate-methacrylic acid (2:1) copolymer), Eudragit LD-55 (an ethylacrylate-methacrylic acid (1:1) copolymer), copolymers of acrylates and methacrylates with quaternary ammonium groups, sold under the tradenames Eudragit RL and Eudragit RS, and a neutral ester dispersion without any functional groups, sold under the tradename Eudragit NE30-D.

Additional shell polymers include: poly(styrene sulfonate), Polycarbophil®; Polyacrylic acid(s); carboxymethyl cellulose, cellulose acetate phthalate, hydroxypropyl methylcellulose phthalate as sold under the tradename HP-50 and HP-55 (Shin-Etsu Chemical Co., Ltd.), cellulose acetate trimellitate, cellulose acetate, cellulose acetate butyrate, cellulose acetate propionate, ethyl cellulose, cellulose derivatives, such as hydroxypropylmethylcellulose, methylcelluose, hydroxylethylcellulose, hydroxyethylmethylcellulose, hydroxylethylethylcelluose and hydroxypropylethylcellulose and cellulose derivatives such as cellulose ethers useful in film coating formulations, polyvinyl acetate phthalate, carrageenan, alginate, or poly(methacrylic acid) esters, acrylic/maleic acid copolymers, styrene/maleic acid polymers, itaconic acid/acrylic copolymers, and fumaric/acrylic acid copolymers, polyvinyl acetal diethylaminoacetate, as sold under the tradename AEA (Sankyo Co., Ltd.), methylvinylether/maleic acid copolymers and shellac.

In some embodiments the shell polymers are selected amongst pharmaceutically acceptable polymers such as Eudragit L100-55 and Eudragit L100 (a methylmethacrylate-methacrylic acid (1:1) copolymer, Degussa/Rohm), Carbopol 934 (polyacrylic acid, Noveon), C-A-P NF (cellulose acetate phthalate—Eastman), Eastacryl (methacrylic acid esters—Eastman), Carrageenan and Alginate (FMC Biopolymer), Anycoat—P (Samsung Fine Chemicals—HPMC Phthalate), or Aqualon (carboxymethyl cellulose—Hercules), methylvinylether/maleic acid copolymers (Gantrez), and styrene/maleic acid (SMA).

The shell can be coated by a variety of methods. In one embodiment, the shell materials are added in the drug formulation step as an active excipient; for example, the shell material can be included in a solid formulation as a powder, which is physically blended with the organophosphate or phosphate-binding polymer and other excipients, optionally granulated, and compressed to form a tablet. Thus, in some embodiments, the shell material need not cover the core material in the drug product. For example, the acidic shell polymer may be added together with the anion binding core polymer formulated in the shape of a tablet, capsule, gel, liquid, etc, wafer, extrudates and the shell polymer can then dissolve and distribute itself uniformly as a shell coating around the core while the drug product equilibrates in the mouth, esophagus or ultimately in the site of action, i.e. the GI tract.

In some embodiments, the shell is a thin layer of shell polymer. The layer can be a molecular layer of polyanion on the core particle surface. The weight to core ratio can be between about 0.0001% to about 30%, preferably comprised between about 0.01% to about 5%, such as between about 0.1% to about 5%.

The shell polymers have a minimum molecular weight such that they do not freely permeate within the core pore volume nor elute from the core surface. In some embodiments, the molecular weight (Mw) of the shell acidic polymer is above about 1000 g/mole, such as above about 5000 g/mole, and or even above about 20,000 g/mole

The anionic charge density of the shell material (as prevailing in the milieu of use) is may be between 0.5 mEq/gr to 22 mEq/gr, such as 2 mEq/gr to 15 mEq/gr. If a coating process is used to form the shell on the polymer particles as part of the manufacture of the dosage form, then procedures known from those skilled-in-the-art in the pharmaceutical industry are applicable. In one embodiment, the shell is formed in a fluidized bed coater (Wurster coater). In an alternate embodiment, the shell is formed through controlled precipitation or coascervation, wherein the polymer particles are suspended in a polymer solution, and the solvent properties are changed in such a way as to induce the polymer to precipitate onto or coat the polymer particles.

Suitable coating processes include the procedures typically used in the pharmaceutical industry. Typically, selection of the coating method is dictated by a number of parameters, that include, but are not limited to the form of the shell material (bulk, solution, emulsion, suspension, melt) as well as the shape and nature of the core material (spherical beads, irregular shaped, etc.), and the amount of shell deposited. In addition, the cores may be coated with one or more shells and may comprise multiple or alternating layers of shells.

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or a severly impaired glomerular filtration rate such as, for example, less than about 20% of normal. The present invention may also be used to treat patients suffering from hyperphosphatemia in End Stage Renal Disease and who are also receiving dialysis treatment (e.g., hemodialysis or peritoneal dialysis).

Other diseases that can be treated with the methods, compounds, compositions, and kits of the present invention include hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues. Also, the present invention can be used to treat Chronic Kidney Disease (CKD), End Stage Renal Disease (ESRD) and dialysis patients, including prophylactic treatment of any of the above.

The polymers, copolymers, polymer networks and/or copolymer networks and compositions described herein can be used as an adjunct to other therapies e.g. those employing dietary control of phosphorus intake, dialysis, inorganic metal salts and/or other polymer resins.

The compositions of the present invention are also useful in removing chloride, bicarbonate, oxalate, and bile acids from the gastrointestinal tract. Polymers, copolymers, polymer networks and/or copolymer networks removing oxalate compounds or ions find use in the treatment of oxalate imbalance disorders, such as oxalosis or hyperoxaluria that increases the risk of kidney stone formation. Polymers, copolymers, polymer networks and/or copolymer networks removing chloride compounds or ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animals. The term “animal” or “animal subject” or “patient” as used herein includes humans as well as other mammals (e.g., in veterinary treatments, such as in the treatment of dogs or cats, or livestock animals such as pigs, goats, cows, horses, chickens and the like). One embodiment of the invention is a method of removing phosphorous-containing compounds such as organophosphates or phosphate from the gastrointestinal tract, such as the stomach, small intestine or large intestine of an animal by administering an effective amount of at least one of the polymers, copolymers, polymer networks and/or copolymer networks described herein.

The term “treating” and its grammatical equivalents as used herein includes achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of polymers, copolymers, polymer networks and/or copolymer networks, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodistrophy. For prophylactic benefit, for example, the polymers, copolymers, polymer networks and/or copolymer networks may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Other embodiments of the invention are directed towards pharmaceutical compositions comprising at least one of the polymers, copolymers, polymer networks and/or copolymer networks or a pharmaceutically acceptable salt thereof, and one or more pharmaceutically acceptable excipients, diluents, or carriers and optionally additional therapeutic agents. The compounds may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the amine polymer with the excipients or carriers and then, if necessary, dividing the product into unit dosages thereof.

The pharmaceutical compositions of the present invention include compositions wherein the polymers, copolymers, polymer networks and/or copolymer networks are present in an effective amount, i.e., in an amount effective to achieve therapeutic and/or prophylactic benefit. The actual amount effective for a particular application will depend on the patient (e.g. age, weight) the condition being treated; and the route of administration.

The dosages of the polymers, copolymers, polymer networks and/or copolymer networks in animals will depend on the disease being, treated, the route of administration, and the physical characteristics of the animal being treated. Such dosage levels in some embodiments for either therapeutic and/or prophylactic uses may be from about 1 gm/day to about 30 gm/day, for example from about 2 gm/day to about 20 gm/day or from about 3 gm/day to about 7 gm/day. The dose of the polymers, copolymers, polymer networks and/or copolymer networks described herein can be less than about 50 gm/day, less than about 40 gm/day, less than about 30 gm/day, less than about 20 gm/day, and less than about 10 gm/day.

Typically, the polymers, copolymers, polymer networks and/or copolymer networks can be administered before or after a meal, or with a meal. As used herein, “before” or “after” a meal is typically within two hours, preferably within one hour, more preferably within thirty minutes, most preferably within ten minutes of commencing or finishing a meal, respectively.

Generally, it is preferred that the polymers, copolymers, polymer networks and/or copolymer networks are administered along with meals. The polymers, copolymers, polymer networks and/or copolymer networks may be administered one time a day, two times a day, or three times a day. Preferably the polymers, copolymers, polymer networks and/or copolymer networks are administered once a day with the largest meal.

Preferably, the polymers, copolymers, polymer networks and/or copolymer networks may be used for therapeutic and/or prophylactic benefits and can be administered alone or in the form of a pharmaceutical composition. The pharmaceutical compositions comprise the polymers, copolymers, polymer networks and/or copolymer networks, one or more pharmaceutically acceptable carriers, diluents or excipients, and optionally additional therapeutic agents. For example, the polymers, copolymers, polymer networks and/or copolymer networks of the present invention may be co-administered with other active pharmaceutical agents depending on the condition being treated. Examples of pharmaceutical agents that may be co-administered include, but are not limited to:

Other phosphate sequestrants including pharmaceutically acceptable lanthanum, calcium, aluminum, magnesium and zinc compounds, such as acetates, carbonates, oxides, hydroxides, citrates, alginates, and ketoacids thereof.

Calcium compounds, including calcium carbonate, acetate (such as PhosLo® calcium acetate tablets), citrate, alginate, and ketoacids, have been utilized for phosphate binding.

Aluminium-based phosphate sequestrants, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient.

The most commonly used lanthanide compound, lanthanum carbonate (Fosrenol®) behaves similarly to calcium carbonate.

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Various examples of pharmaceutically acceptable zinc compounds are described in PCT Application No. PCT/US2005/047582 filed Dec. 29, 2005, the entire teachings of which are incorporated herein by reference. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in the invention in combination with pharmaceutically acceptable ferrous iron salts.

In other embodiments, the phosphate sequestrant used in combination with polymers, copolymers, polymer networks and/or copolymer networks of the present invention is not a pharmaceutically acceptable magnesium compound. In yet other embodiments, the phosphate sequestrant used in combination with the pharmaceutically acceptable polymers, copolymers, polymer networks and/or copolymer networks is not a pharmaceutically acceptable zinc compound.

The invention also includes methods and pharmaceutical compositions directed to a combination therapy of the polymers, copolymers, polymer networks and/or copolymer networks in combination with a phosphate transport inhibitor or an alkaline phosphatase inhibitor. Alternatively, a mixture of the polymers, copolymers, polymer networks and/or copolymer networks is employed together with a phosphate transport inhibitor or an alkaline phosphatase inhibitor.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

A large variety of organic and inorganic molecules are inhibitors to alkaline phosphatase (ALP) (see, for example, U.S. Pat. No. 5,948,630, the entire teachings of which are incorporated herein by reference). Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl) imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the polymers, copolymers, polymer networks and/or copolymer networks may be co-administered with calcium salts which are used to treat hypocalcemia resulting from hyperphosphatemia.

The pharmaceutical compositions of the invention can be formulated as a tablet, sachet, slurry, food formulation, troche, capsule, elixir, suspension, syrup, wafer, chewing gum or lozenge.

Preferably, the polymers, copolymers, polymer networks and/or copolymer networks or the pharmaceutical compositions comprising the polymers, copolymers, polymer networks and/or copolymer networks is administered orally. Illustrative of suitable methods, vehicles, excipients and carriers are those described, for example, in Remington's Pharmaceutical Sciences, 19th ed., the contents of which is incorporated herein by reference.

Pharmaceutical compositions for use in accordance with the present invention may be formulated in conventional manner using one or more physiologically acceptable carriers comprising excipients and auxiliaries which facilitate processing of the active polymers, copolymers, polymer networks and/or copolymer networks into preparations which can be used pharmaceutically. Proper formulation is dependent upon the route of administration chosen. Suitable techniques for preparing pharmaceutical compositions of the amines are well known in the art.

In some aspects of the invention, the polymers, copolymers, polymer networks and/or copolymer networks provide mechanical and thermal properties that are usually performed by excipients, thus decreasing the amount of such excipients required for the formulation. In some embodiments the polymers, copolymers, polymer networks and/or copolymer networks constitutes over about 30 wt. %, for example over about 40 wt. %, over about 50 wt. %, preferably over about 60 wt. %, over about 70 wt. %, more preferably over about 80 wt. %, over about 85 wt. % or over about 90 wt. % of the composition, the remainder comprising suitable excipient(s).

In some embodiments, the compressibility of the tablets is strongly dependent upon the degree of hydration (moisture content) of the polymers, copolymers, polymer networks and/or copolymer networks. Preferably, the polymers, copolymers, polymer networks and/or copolymer networks has a moisture content of about 5% by weight or greater, more preferably, the moisture content is from about 5% to about 9% by weight, and most preferably about 7% by weight. It is to be understood that in embodiments in which the amine polymer is hydrated, the water of hydration is considered to be a component of the amine polymer.

The tablet can further comprise one or more excipients, such as hardeners, glidants and lubricants, which are well known in the art. Suitable excipients include colloidal silicon dioxide, stearic acid, magnesium silicate, calcium silicate, sucrose, calcium stearate, glyceryl behenate, magnesium stearate, talc, zinc stearate and sodium stearylfumarate.

The tablet core of embodiments of the invention may be prepared by a method comprising the steps of: (1) hydrating or drying the polymers, copolymers, polymer networks and/or copolymer networks to the desired moisture level; (2) blending the polymers, copolymers, polymer networks and/or copolymer networks with any excipients; and (3) compressing the blend using conventional tableting technology.

In some embodiments, the invention relates to a stable, swallowable coated tablet, particularly a tablet comprising a hydrophilic core, such as a tablet comprising the polymers, copolymers, polymer networks and/or copolymer networks, as described above. In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating, a white pigment can be selected, such as titanium dioxide.

In one embodiment, the coated tablet of the invention can be prepared by a method comprising the step of contacting a tablet core of the invention, as described above, with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in the some compositions of the invention include a binder, such as microcrystalline cellulose, carbopol, providone and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (ed), Remington's Pharmaceutical Sciences, 19th Edition.

In some embodiments the polymers, copolymers, polymer networks and/or copolymer networks of the invention are provided as pharmaceutical compositions in the form of chewable tablets. In addition to the active ingredient, the following types of excipients are commonly used: a sweetening agent to provide the necessary palatability, plus a binder where the former is inadequate in providing sufficient tablet hardness; a lubricant to minimize frictional effects at the die wall and facilitate tablet ejection; and, in some formulations a small amount of a disintegrant is added to facilitate mastication. In general excipient levels in currently-available chewable tablets are on the order of 3-5 fold of active ingredient(s) whereas sweetening agents make up the bulk of the inactive ingredients. In some embodiments the invention provides a pharmaceutical composition formulated as a chewable tablet, comprising a polymer, copolymer, polymer network and/or copolymer networks described herein, a filler, and a lubricant. In some embodiments the invention provides a pharmaceutical composition formulated as a chewable tablet, comprising a polymer, copolymer, polymer network and/or copolymer network described herein, a filler, and a lubricant, wherein the filler is chosen from the group consisting of sucrose, mannitol, xylitol, maltodextrin, fructose, and sorbitol, and wherein the lubricant is a magnesium fatty acid salt, such as magnesium stearate.

In one embodiment, the polymer, copolymer, polymer network and/or copolymer network is pre-formulated with a high Tg/high melting point low molecular weight excipient such as mannitol, sorbose, sucrose in order to form a solid solution wherein the polymer and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the polymers, copolymers, polymer networks and/or copolymer networks of the invention are provided as pharmaceutical compositions in the form of liquid formulations. In some embodiments the pharmaceutical composition contains a polymer, copolymer, polymer network and/or copolymer network dispersed in a suitable liquid excipient. Suitable liquid excipients are known in the art; see, e.g., Remington's Pharmaceutical Sciences.

In some embodiments, the pharmaceutical compositions may be in the form of a powder formulation packaged as a sachet that may be mixed with water or other ingestible liquid and administered orally as a drink (solution or suspension). In order to ensure that such formulations provide acceptable properties to the patient such as mouth feel and taste, a pharmaceutically acceptable anionic stabilizer may be included in the formulation.

Examples of suitable anionic stabilizers include anionic polymers such as: an anionic polypeptide, an anionic polysaccharide, or a polymer of one or more anionic monomers such as polymers of mannuronic acid, guluronic acid, acrylic acid, methacrylic acid, glucuronic acid glutamic acid or a combination thereof, and pharmaceutically acceptable salts thereof. Other examples of anionic polymers include cellulose, such as carboxyalkyl cellulose or a pharmaceutically acceptable salt thereof. The anionic polymer may be a homopoloymer or copolymer of two or more of the anionic monomers described above. Alternatively, the anionic copolymer may include one or more anionic monomers and one or more neutral comonomers such as olefinic anionic monomers such as vinyl alcohol, acrylamide, and vinyl formamide.

Examples of anionic polymers include alginates (e.g. sodium alginate, potassium alginate, calcium alginate, magnesium alginate, ammonium alginate, and esters of alginate), carboxymethyl cellulose, polylactic acid, polyglutamic acid, pectin, xanthan, carrageenan, furcellaran, gum Arabic, karaya gum, gum ghatti, gum carob, and gum tragacanth. Preferred anionic polymers are alginates and are preferably esterified alginates such as a C2-C5-diol ester of alginate or a C3-C5 triol ester of alginate. As used herein an “esterified alginate” means an alginic acid in which one or more of the carboxyl groups have of the alginic acid are esterified. The remainder of the carboxylic acid groups in the alginate are optionally neutralized (partially or completely) as pharmaceutically acceptable salts. For example, propylene glycol alginate is an ester of alginic acid in which some of the carboxyl groups are esterified with propylene glycol, and the remainder of the carboxylic acid groups are optionally neutralized with pharmaceutically acceptable salts. More preferably, the anionic polymer is ethylene glycol alginate, propylene glycol alginate or glycerol alginate, with propylene glycol alginate even more preferred.

All publications and patent applications mentioned in this specification are herein incorporated by reference to the same extent as if each individual publication or patent application was specifically and individually indicated to be incorporated by reference.

It will be apparent to one of ordinary skill in the art that many changes and modification can be made to the disclosures presented herein without departing from the spirit or scope of the appended claims.

### EXAMPLES

### Materials Used

Divinyl sulfone, N-methyl-1,3-propane diamine, 4-(aminomethyl)-piperidine, chloroform, epichlorohydrin, methanol and acetone are commercially available from Sigma-Aldrich, Co.

### Example 1

### Synthesis of Compound

A flask was charged with 15.06 ml of divinyl sulfone, 15.52 ml of N-methyl-1,3 propane diamine and 60 ml of chloroform. The mixture exothermed to 59° C., and then was heated at 40° C. for 96 hours with stirring. The mixture was cooled to room temperature and poured into a solution of 2.5 L of methanol and 50 ml of concentrated HCl. The precipitate was collected by filtration, suspended in a hot solution of 50% (v/v) methanol and acetone, stirred for 10 min and filtered. The precipitate was re-suspended in a hot solution of 50% (v/v) methanol and acetone, stirred for 10 min and filtered. The resulting material was dried in a vacuum oven at 70° C. with a small bleed of nitrogen gas to yield 30.20 g of the desired product.

### Examples 2

### Synthesis of Compounds II-IV

Using the procedure described for Example 1, Compounds II-IV were synthesized as indicated in Table I:

TABLE I
Synthesis of Compounds II-IV

| Compound | | Multifunctional Amine- | | |
|---|---|---|---|---|
| Synthesized | Amine (amount) | Reactive Compound (amount) | Solvent (amount) | Yield |
| II | N-methyl-1,3-propane | Divinyl sulfone (15.06 ml) | Chloroform (60 ml) | 31.36 |
| | diamine (15.52 ml) | | | |
| III | 4-(aminomethyl)- | Divinyl sulfone (15.06 ml) | Chloroform (60 ml) | 33.86 |
| | piperidine (17.99 ml) | | | |
| IV | 1-(2-aminoethyl)- | Divinyl sulfone (15.06 ml) | Chloroform (60 ml) | 37.83 |
| | piperazine (19.68 ml) | | | |

### Example 3

### Reaction of Compound I with Epichlorohydrin

8.0 g of a 50% aqueous solution of NaOH was added to a solution of 15 g of Compound I in 15 g of deionized water. The resulting solution had a pH of 10.29. 0.569 ml of epichlorohydrin was added to this solution, stirred overnight at room temperature, and then heated to 60° C. for 1 hour. No gel was observed. A second portion of 0.569 ml of epichlorohydrin was added and the solution was heated in a closed container at 60° C. overnight. The resulting gel was broken into small pieces suspended in 2 L of deionized water, stirred for 20 min and filtered. The filtered material was re-suspended in 2 L of deionized water, stirred for 20 min and filtered. Prior to filtering the suspension had a conductivity of 70.3 us/cm. The filtered material, having a wet weight of 22.07 g was dried in a forced air oven at 60° C. to afford 2.68 g of rubbery material. The rubbery material was suspended in deionized water and the pH was adjusted with HCl to 1. The material was filtered and dried in a forced air oven at 60° C. to afford 3.51 g of the desired product.

### Example 4

### Dialysis of Compound I

A solution of 10 g of Compound I in 50 ml of deionized water was dialyzed against deionized water using MWCO 3500 tubing, until the conductivity was 17.9 uS/cm. The dialyzed material was lyophilized to afford 2.27 g.

### Example 5

### Reaction of Compound IV with Epichlorohydrin

9.1 g of a 50% aqueous solution of NaOH was added to a pH 3.2 solution of 15 g of Compound IV in 30 g of deionized water. The resulting solution had a pH of 10.51. 0.474 ml of epichlorohydrin was added to this solution and stirred overnight at room temperature. No gel was observed. A second portion of 0.474 ml of epichlorohydrin was added and the solution was stirred at room temperature for 6 hours and heated in a closed container at 60° C. for 2 hours. No gel was observed. A third portion of 0.474 ml of epichlorohydrin was added and the solution was stirred overnight at room temperature and heated in a closed container at 60° C. for 5 hours. No gel was observed. A fourth portion of 0.474 ml of epichlorohydrin was added and the solution was stirred overnight at room temperature. No gel was observed. A fifth portion of 0.474 ml of epichlorohydrin was added and the solution was heated in a closed container at 60° C. overnight. The resulting gel was broken into small pieces suspended in 2 L of deionized water, stirred for 20 min and filtered. The filtered material was re-suspended in 2 L of deionized water, stirred for 20 min and filtered. The filtered material was suspended in 2 L of deionized water and had the suspension had a conductivity of 0.31 mS/cm and a pH of 6.2. 55.3 ml of concentrated HCL was added to the suspension to adjust the pH to 1.03. The suspension was filtered and the filtered material, having a wet weight of 127.39 g, was dried in a forced-air oven at 60° C. to afford 10.83 g of the desired product having an In-Process Swelling Ratio of 10.76 ml/g.

### Test Methods

### Urinary Phosphorous Reduction (In Vivo—Rats)

House male Sprague Dawley (SD) rats may be used for the experiments. The rats are placed singly in wire-bottom cages, fed with Purina 5002 diet, and allowed to acclimate for at least 5 days prior to experimental use.

To establish baseline phosphorus excretion, the rats are placed in metabolic cages for 48 hours. Their urine is collected and its phosphorus content analyzed with a Hitachi analyzer to determine phosphorus excretion in mg/day. Any rats with outlying values are excluded; and the remainder of the rats are distributed into groups.

Purina 5002 is used as the standard diet. The active being tested is mixed with Purina 5002 to result in a final concentration of 0.25% by weight of the feed. Cellulose at 0.5% by weight is used as a negative control. Sevelamer at 0.5% by weight is used as a positive control. For each rat, 200 g of diet is prepared.

Each rat is weighed and placed on the standard diet. After 4 days the standard diet is replaced with the treatment diet (or control diet for the control group). On days 5 and 6, urine samples from the rats at 24 hours (+/−30 minutes) are collected and analyzed. The test rats are again weighed, and any weight loss or gain is calculated. Any remaining food is also weighed to calculate the amount of food consumed per day. A change in phosphorus excretion relative to baseline and cellulose negative control is calculated. Percentage reduction of urinary phosphorous may be determined by the following equation:

% Reduction of Urinary Phosphorous=[(urinary phosphorous of negative control(mg/day)−urinary phosphorous of experimental(mg/day))/urinary phosphorous of negative control(mg/day)]×100.

In Vitro Phosphate Binding (mmol/g)

Two samples per polymer are weighed into plastic bottles after having adjusted the weight of the polymer for the loss on drying of each sample. A 10 mM phosphate buffer solution containing 10 mM KH₂PO₄, 100 mM N,N-bis[2-hydroxyethyl]-2-aminoethanesulfonic acid, 80 mM NaCl, 15 mM glycochenodeoxycholic acid (GCDC), and 15 mM oleic acid (pH adjusted to 7.0 with 1 N NaOH) is prepared and well mixed. Aliquots of the 10 mM phosphate buffer solution is transferred into each of the two sample bottles. The solutions are well mixed and then placed into an orbital shaker at 37° C. for 1 hour. The polymer is allowed to settle prior to removing a sample aliquot from each solution. The sample aliquot is filtered into a small vial using a disposable syringe and syringe filter. The filtered sample is diluted 1-to-10 with DI water. The shaking is continued for a further 4 hours (total of 5 hours) and the sampling procedure is repeated. Phosphate standards are prepared from a 10 mM phosphate standard stock solution and diluted appropriately to provide standards in the range of 0.3 to 1.0 mM. Both the standards and samples are analyzed by ion chromatography. A standard curve is set up and the unbound phosphate (mM) for each test solution is calculated. Bound phosphate is determined by the following equation:

Bound Phosphate(mmol/g)=[(10−Unbound PO₄)×Vol.×1000]/MassP; wherein Vol.=volume of test solution (L); MassP=LOD adjusted mass of polymer (mg).

In-Process Swelling Ratio (mL/g)

The in-process swelling ratio (SR) is determined by the following equation:

SR=(weight of wet gel(g)−weight of dry polymer(g))/weight of dry polymer(g).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-30. (canceled)

31. A pharmaceutical composition comprising:
a) a hyperbranched copolymer derived from:
(i) a multi-amine monomer comprising at least one secondary amine;
(ii) a vinyl sulfonyl-containing monomer; and
b) a pharmaceutically acceptable excipient.

32. The composition according to claim 31, wherein the vinyl sulfonyl-containing monomer is selected from the group consisting of: 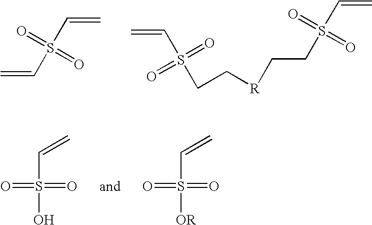
wherein R independently represents a branched or unbranched, substituted or unsubstituted alkyl radical.

33. The composition according to claim 31, wherein said copolymer has a degree of branching of from 0.25 to 0.75.

34. The composition according to claim 31, wherein from 25-75% of the nitrogen atoms in the copolymer are the nitrogen in a secondary amine moiety.

35. The composition according to claim 31, wherein said copolymer has a polydispersity greater than about 1.5.

36. The composition according to claim 31, wherein greater than 25% and less than 75% of non-terminal, non-amido amine groups in said copolymer comprise tertiary amines.

37. The pharmaceutical composition according to claim 31, wherein the multi-amine monomer is selected from: 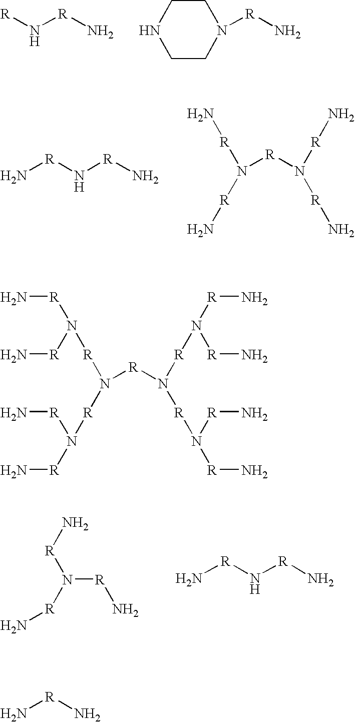
and combinations thereof.

38. The pharmaceutical composition according to claim 31, wherein the multi-amine monomer includes at least one protected amine group.

39. The pharmaceutical composition according to claim 38, wherein the protected amine group is protected with a tert-butyloxycarbonyl group.

40. The pharmaceutical composition according to claim 31, wherein the copolymer further comprises a second multi-amine or residue thereof.

41. The composition according to claim 40, wherein the second multi-amine is selected from the group consisting of:

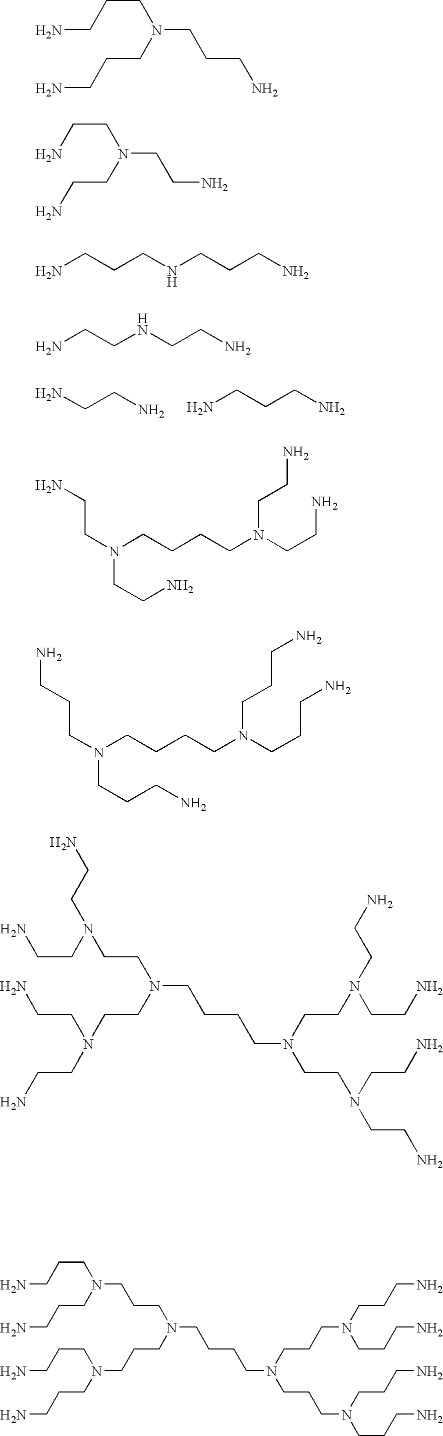

42. A pharmaceutical composition comprising:
a) a copolymer derived from compounds according to the following Formulas I and II: 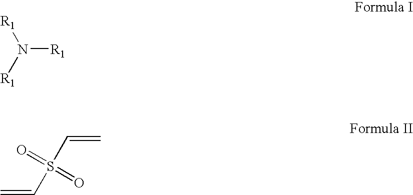
wherein
where R₁ independently represents a hydrogen radical, —R or —R—N(H)₂₋ₘ—(R—N(H)₂₋ₙ—(R—NH₂)ₙ)ₘ or R₁ and another R₁ combine to form a heterocyclic ring;
n and m independently represent an integer from 0 to 2;
R independently represents a branched or unbranched, substituted or unsubstituted alkyl radical, with the proviso that at least one R₁ is not a hydrogen radical or —R; and
b) a pharmaceutically acceptable excipient.

43. The pharmaceutical composition according to claim 42, wherein the compound according to Formula I comprises a combination of tris(2-aminoethyl)amine and tris(3-aminopropyl)amine.

44. The composition according to claim 42, wherein said copolymer has a degree of branching of from 0.25 to 0.75.

45. The composition according to claim 42, wherein from 25-75% of the nitrogen atoms in the copolymer are the nitrogen in a secondary amine moiety.
